(19) United States
(12) Patent Application Publication
(10) Pub. No.: US 2024/0233950 A1
(43) Pub. Date: Jul. 11, 2024

(54) Systems and Methods for Computing Measurements for Mitochondrial Diseases

(71) Applicants: RIBONOVA, INC., Wynnewood, PA (US); The Children's Hospital of Philadelphia, Philadelphia, PA (US)

(72) Inventors: Marni J. Falk, Philadelphia, PA (US); Nigel L. Webb, Wynnewood, PA (US); Shana E. McCormack, Philadelphia, PA (US); Eric C. Yuen, Wynnewood, PA (US); Zarazuela Zolkipli-Cunningham, Philadelphia, PA (US); Anthony W. Ford-Hutchinson, Wynnewood, PA (US)

(73) Assignees: RIBONOVA, INC., Wynnewood, PA (US); The Children's Hospital of Philadelphia, Philadelphia, PA (US)

(21) Appl. No.: 18/417,247

(22) Filed: Jan. 19, 2024

Related U.S. Application Data
(63) Continuation of application No. 17/255,531, filed on Dec. 23, 2020, now abandoned.
(60) Provisional application No. 62/690,018, filed on Jun. 26, 2018.

(51) Int. Cl. G16H 50/30; G16B 20/00; G16H 10/20; G16H 10/40; G16H 10/60; G16H 50/20; G16H 50/70

(52) U.S. Cl. CPC G16H 50/30; G16B 20/00; G16H 10/20; G16H 10/40; G16H 10/60; G16H 50/20; G16H 50/70

(57) ABSTRACT

This disclosure is directed to methods of methods of computing a composite measurement comprising employing two or more sub-instruments to measure one or more clinical symptoms of mitochondrial dysfunction or mitochondrial disease. This disclosure is also directed to methods of assessing and managing a subject with mitochondrial dysfunction or mitochondrial disease using a composite measurement.

### TECHNICAL FIELD

Systems and methods are provided for computing composite measurements for assessment and management of mitochondrial dysfunction or mitochondrial disease in a human subject, wherein the composite instrument combines the results from two or more sub-instruments that measure one or more clinical symptoms of mitochondrial dysfunction or mitochondrial disease in the subject.

### BACKGROUND

Primary mitochondrial disease encompasses a heterogeneous group of genetic conditions that impair the ability to generate cellular energy. Kremer et al., “Mitochondrial Disease Genetics” in Diagnosis and Management of Mitochondrial Disorders; Mancuso and Klopstock (Eds); Springer 2019; pp 41-62. The disease affects at least 1 in 4,300 individuals with a highly variable, but often progressive array of multi-systemic manifestations. Gorman et al., Ann. Neurol. 2015; 77:753-9. The more severe manifestations can lead to significant morbidity and mortality. Two studies of affected children found mortality rates of 35% and 36%, respectively, during their childhood years. Scaglia et al., Pediatrics. 2004; 114:925-31. Verity et al., Dev. Med. Child Neurol. 2010; 52:434-40. In a longitudinal study of all non-military hospitalizations in California from 2007 to 2011, 9.9% of all participants (9% of adults, 10% of children) with at least one hospitalization for mitochondrial disease died in-hospital during five years of follow-up. McCormack et al., Mol. Genet. Metab. 2017; 121(2):119-126. There are no proven treatment options for the underlying disease process. Pfeffer et al., Nat. Rev. Neurol. 2013; 9:474-81. Thus, there is an unmet medical need for diagnosing, preventing, and treating mitochondrial disease.

### SUMMARY

The invention relates to a method of computing a composite measurement for the assessment, diagnosis, monitoring or treatment of a subject with mitochondrial dysfunction or mitochondrial disease, said method comprising (i) employing a panel of two or more sub-instruments to measure one or more clinical symptoms of mitochondrial dysfunction or mitochondrial disease in a subject, (ii) combining the measurements obtained from said two or more sub-instruments into a single composite measurement, and (iii) assessing the overall severity of, or change in, the mitochondrial dysfunction or mitochondrial disease in the subject by comparing the composite measurement to a reference value or another composite measurement in the same subject.

In some embodiments, the one or more composite measurements are employed to measure the clinical effect on the subject of a diagnostic, therapeutic or other type of medical intervention, wherein (i) for each of the sub-instruments in said panel, the subject is classified as (a) a sub-instrument responder or sub-instrument non-responder, (b) a member of a clinical category, or (c) a member of a metric range, based on the change in said one or more clinical symptoms as measured using said sub-instrument, and (ii) the measurements obtained from the sub-instruments in the panel are combined into a single composite measurement, by either: (a) separately assessing the change in each measurement obtained from the panel sub-instruments prior to combining each measurement into a single composite measurement, or (b) combining measurements obtained from the panel sub-instruments at a first time point and generating a single composite measurement for said first time point and then comparing the single composite measurement for said first time point to a single composite measurement generated from the same panel sub-instruments for a second time point.

In some embodiments, the panel sub-instruments comprise one or more of Motor Function Measure, Six Minute Walk Test, Two Minute Walk Test, Modified Fatigue Impact Scale, Friedreich's Ataxia Rating Scale, Patient-Reported Outcomes Measurement Information System Gastrointestinal Symptom Scales, 30-Second Chair Stand Test, Muscle Strength by Myometry Test, Newcastle Mitochondrial Disease Adult Scale, Newcastle Mitochondrial Disease Pediatric Scale, 36-Item Short Form Survey, Clinical Global Impression, Patient's Global Impression, Patient-Reported Initial MitoPC Symptoms, Patient-Reported MitoPC Symptom Changes, Columbia Suicide Severity Rating Scale, Neuropathy Impairment Score, Migraine Disability Assessment Test, Quick Inventory of Depressive Symptomatology—Self-Report, Montreal Cognitive Assessment, NY Heart Association Functional Classification, Diabetes Health Profile, Ocular Motility Assessments, Marginal Reflex Distance, Ocular Motility & Fixation in 8 Gaze Directions, Degree of Ocular Saccades, Visual Function Test, Logarithm of Minimum Angle of Resolution, Pelli-Robson Score, Humphrey Visual Field mean deviation, or a combination thereof.

In some embodiments, the panel sub-instruments comprise Motor Function Measure, Six Minute Walk Test, Modified Fatigue Impact Scale, Friedreich's Ataxia Rating Scale, and the Patient-Reported Outcomes Measurement Information System Gastrointestinal Symptom Scales. In some embodiments, the panel sub-instruments comprises five sub-instruments, including but not limited to, Motor Function Measure, Six Minute Walk Test, Modified Fatigue Impact Scale, Friedreich's Ataxia Rating Scale, and the Patient-Reported Outcomes Measurement Information System Gastrointestinal Symptom Scales.

### DETAILED DESCRIPTION

The disclosed methods will be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods.

In some aspects, methods of computing a composite measurement are disclosed for the assessment, diagnosis or treatment of subjects with potential or actual mitochondrial dysfunction or mitochondrial disease, wherein the methods comprise: (i) employing two or more sub-instruments to measure one or more clinical symptoms of mitochondrial dysfunction or mitochondrial disease in a subject, and (ii) combining the measurements obtained from the two or more sub-instruments into a single composite measurement.

In certain embodiments, the composite measurement is employed to measure one or more clinical symptoms, and a change in the composite measurement of clinical symptoms is compared to a baseline composite measurement, at a single point in time. In certain embodiments, changes in the composite measurement of clinical symptoms are compared to a baseline composite measurement over a time period.

In some embodiments, disclosed herein are methods of managing patients with mitochondrial dysfunction or mitochondrial disease and/or assessing the response of these patients to therapeutic intervention through the use of a composite instrument that measures clinically significant improvements, comprising (i) a panel of multiple sub-instruments that measure relevant and/or prevalent symptoms affecting patients with mitochondrial disease; (ii) a baseline assessment of a patient using a composite measurement from the sub-instrument results; (iii) a subsequent composite assessment of a patient using the same sub-instruments; and (iv) a comparison of the result of the subsequent composite assessment to the baseline composite assessment.

In some embodiments, (i) for each of the two or more sub-instruments, the subject is classified as (a) a sub-instrument responder or sub-instrument non-responder, (b) a member of a clinical category, or (c) a member of a metric range, based on the change in the one or more clinical symptoms as measured using the sub-instrument, and (ii) the measurements obtained from the two or more sub-instruments are combined into a single composite measurement, wherein the combination comprises (a) separately assessing the change in each measurement obtained from the two or more sub-instruments prior to combining each measurement into a single composite measurement, or (b) combining measurements obtained from the two or more sub-instruments at a first time point and generating a single composite measurement for the first time point and then comparing the single composite measurement for the first time point to a single composite measurement generated from the same two or more sub-instruments for a second time point. Representative computations are detailed in the Examples provided herein.

By way of example, classifying the subject as a member of a clinical category may mean classifying the subject as having a normal or abnormal measurement.

By way of further example, classifying the subject as a member of a metric range may mean classifying the subject as falling within or outside a pre-defined numeric range.

In some embodiments, determining a composite endpoint entails the use of two or more distinct endpoints (the component endpoints), the results from which are used to compute an overall result. Composite endpoints are employed to measure the progression of patients with a variety of diseases and disorders, in which the subjects exhibit multiple clinical symptoms that reflect distinct bodily dysfunctions. Overall patient improvement or deterioration is often challenging to measure without the use of such composite endpoints. In order to achieve an adequate statistical power for a clinical study, investigators frequently combine the results from two or more endpoints to derive an overall composite event rate. McCoy, West J Emerg Med. 2018; 19(4): 631-634. The derived composite endpoints can provide a broader picture of patient function or dysfunction, and enable a more accurate assessment of overall clinical improvement or deficit. Examples of established composite endpoints include the Patient-Reported Outcome Measurement Information System (PROMIS), the Multiple Sclerosis Quality of Life and Depression (MSQOL), the Multiple Sclerosis Functional Composite (MSFC), the Unified Parkinson's Disease Ratings Scale (UPDRS) and the Expanded Disability Status Scale (EDSS).

Mitochondrial diseases exhibit considerable clinical variability from patient to patient. The Newcastle Mitochondrial Disease Adult Scale (NMDAS) and the Newcastle Paediatric Mitochondrial Disease Scale (NPMDS) have been proposed as composite scales to measure the progression of patients with mitochondrial disease. These two similar semi-quantitative clinical rating scales were designed to measure all forms of mitochondrial disease, in adult and pediatric subjects, respectively. Their purpose was to provide a validated and reproducible measurement of disease progression over time to support clinical research and treatment trials. Schaefer et al., Neurology 2006; 66(12): 1932-1934; Phoenix et al. Neuromuscular Disorders 2006; 16: 814-820.

In contrast to the composite instruments described herein, the NMDAS and NPMDS were designed to measure progression of mitochondrial disease, and neither is intended to be used as a composite endpoint to assess the response to medical intervention. Additionally, both NMDAS and NPMDS involve assessments of physical functions by a person (examiner) administering the test and they are not direct measures of physical functions. NMDAS and NPMDS were designed by and for neurologists and require that the examiner have extensive experience with care of mitochondrial disease patients, and broad previous exposure to the full range of manifestations of mitochondrial disease. NMDAS and NPMDS were also designed to be administered at six or twelve month intervals, and thus were not designed for more rapid assessment of responses to clinical interventions.

The composite instruments in accordance with embodiments of the present disclosure are used to assess patient response to medical intervention. They utilize a set of established, validated, objective measurements of physical function and symptoms, allowing for greater granularity of clinical measurement and administration by easily trained personnel.

In some embodiments, the mitochondrial dysfunction is respiratory chain dysfunction. Respiratory chain dysfunction, sometimes referred to as respiratory chain deficiency, represents a heterogeneous and highly morbid group of energy deficiency disorders involving aberrations in the cellular oxidative phosphorylation processes.

In some embodiments, the mitochondrial dysfunction is associated with inherited mitochondrial disease. In some embodiments, the mitochondrial dysfunction is an acquired mitochondrial disease. In some embodiments, the mitochondrial dysfunction is a primary mitochondrial disease. In some embodiments, the mitochondrial dysfunction is a secondary mitochondrial disease.

In some embodiments, the mitochondrial dysfunction is associated with one or more genetic mutations or variants, sometimes referred to as genetically-confirmed mitochondrial disease. Genetically-confirmed mitochondrial disease represents a class of disorders associated with a wide range of clinical and pathological features. Gorman et al., Nat. Rev. Dis. Prim. 2016; 2:16080. Symptoms are highly varied, and individuals can present at any age, from before birth into older adulthood. Parikh et al., Genet. Med. 2015; 17:689-701.

In some embodiments in accordance with the present disclosure, the subject has a mitochondrial disease selected from autosomal dominant optic atrophy (or “ADOA”); beta-oxidation defects; carnitine deficiency; carnitine-acyl-carnitine deficiency; chronic progressive external ophthalmoplegia syndrome (or “CPEO”); co-enzyme Q10 deficiency; complex I deficiency (or “NADH dehydrogenase deficiency”); complex II deficiency (or “succinate dehydrogenase deficiency”); complex III deficiency (or “ubiquinone-cytochrome c oxidoreductase deficiency”); complex IV deficiency (or “cytochrome c oxidase deficiency” or “COX deficiency”); complex V deficiency (or “ATP synthase deficiency”); multiple respiratory chain complex deficiency; CPT I deficiency; CPT II deficiency; diabetes mellitus and deafness (or “DAD”); creatine deficiency syndrome; Friedreich's ataxia (or “FA”); Kearns-Sayre syndrome (or “KSS”); lactic acidosis; Leber's hereditary optic neuropathy (or “LHON”); Leigh syndrome (or “Leigh disease”); lethal infantile cardiomyopathy (or “LIC” or “Barth Syndrome”); leukodystrophy; leukoencephalopathy with brain stem and spinal cord involvement and lactate elevation (or “LBSL”); long-chain acyl-CoA dehydrogenase deficiency (or “LCAD”); long-chain 3-hydroxyacyl-CoA dehydrogenase (or “LCHAD”); Luft disease; medium-chain acyl-CoA dehydrogenase deficiency (or “MCAD”); mitochondrial cytopathy; mitochondrial DNA depletion; mitochondrial DNA deletion(s); mitochondrial encephalopathy; mitochondrial myopathy (or “MM”); mitochondrial myopathy, encephalomyopathy, lactic acidosis, stroke-like symptoms (or “MELAS”); mitochondrial neurogastrointestinal encephalomyopathy (or “MNGIE”; mitochondrial recessive ataxia syndrome (or “MIRAS”); multiple acyl-CoA dehydrogenase deficiency (or “MAD” or “glutaric aciduria type II”); myoclonic epilepsy with ragged red fibers (or “MERRF”); neuropathy, ataxia, and retinitis pigmentosa (or “NARP”); Pearson syndrome; POLG2 mutations; progressive infantile poliodystrophy (or “Alper's disease”); ptosis; pyruvate carboxylase deficiency (or “PCD”); pyruvate dehydrogenase complex deficiency (or “PDCD” or “PDH”); short-chain acyl-CoA dehydrogenase deficiency (or “SCAD”); short chain 3-hydroxyacyl-CoA dehydrogenase deficiency (or “SCHADD”); very long-chain acyl-CoA dehydrogenase deficiency (or “VLCAD”); or a combination thereof.

In other embodiments of the disclosure the subject has been diagnosed with a mitochondrial disease selected from autosomal dominant optic atrophy (or “ADOA”); beta-oxidation defects; carnitine deficiency; carnitine-acyl-carnitine deficiency; chronic progressive external ophthalmoplegia syndrome (or “CPEO”); co-enzyme Q10 deficiency; complex I deficiency (or “NADH dehydrogenase deficiency”); complex II deficiency (or “succinate dehydrogenase deficiency”); complex III deficiency (or “ubiquinone-cytochrome c oxidoreductase deficiency”); complex IV deficiency (or “cytochrome c oxidase deficiency” or “COX deficiency”); complex V deficiency (or “ATP synthase deficiency”); multiple respiratory chain complex deficiency; CPT I deficiency; CPT II deficiency; diabetes mellitus and deafness (or “DAD”); creatine deficiency syndrome; Friedreich“s ataxia (or “FA”); Kearns-Sayre syndrome (or “KSS”); lactic acidosis; Leber's hereditary optic neuropathy (or “LHON”); Leigh syndrome (or “Leigh disease”); lethal infantile cardiomyopathy (or “LIC” or “Barth Syndrome”); leukodystrophy; leukoencephalopathy with brain stem and spinal cord involvement and lactate elevation (or “LBSL”); long-chain acyl-CoA dehydrogenase deficiency (or “LCAD”); long-chain 3-hydroxyacyl-CoA dehydrogenase (or “LCHAD”); Luft disease; medium-chain acyl-CoA dehydrogenase deficiency (or “MCAD”); mitochondrial cytopathy; mitochondrial DNA depletion; mitochondrial DNA deletion(s); mitochondrial encephalopathy; mitochondrial myopathy (or “MM”); mitochondrial myopathy, encephalomyopathy, lactic acidosis, stroke-like symptoms (or “MELAS”); mitochondrial neurogastrointestinal encephalomyopathy (or “MNGIE”); mitochondrial recessive ataxia syndrome (or “MIRAS”); multiple acyl-CoA dehydrogenase deficiency (or “MAD” or “glutaric aciduria type II”); myoclonic epilepsy with ragged red fibers (or “MERRF”); neuropathy, ataxia, and retinitis pigmentosa (or “NARP”); Pearson syndrome; POLG2 mutations; progressive infantile poliodystrophy (or “Alper's disease”); ptosis; pyruvate carboxylase deficiency (or “PCD”); pyruvate dehydrogenase complex deficiency (or “PDCD” or “PDH”); short-chain acyl-CoA dehydrogenase deficiency (or “SCAD”); short chain 3-hydroxyacyl-CoA dehydrogenase deficiency (or “SCHADD”); very long-chain acyl-CoA dehydrogenase deficiency (or “VLCAD”); or a combination thereof.

In certain embodiments, the change, improvement or delayed onset of one or more clinical symptoms is clinically significant. By “clinically significant,” it is meant to include one or more improvements that one of ordinary skill in the art would consider (i) statistically significant, or (ii) practically important in that it has an observable positive effect on daily life. For example, clinically significant improvements can be improvements of pre-selected symptoms, as compared to pre-defined severity thresholds. In some embodiments, a clinically significant improvement of select symptoms includes the absence of statistically significant deterioration in any of other symptoms. In other embodiments, a clinically significant improvement includes an improvement in a quality of life parameter.

In some aspects, the two or more measurement instruments are selected from Motor Function Measure, Six Minute Walk Test, Two Minute Walk Test, Modified Fatigue Impact Scale, Friedreich's Ataxia Rating Scale, Patient-Reported Outcomes Measurement Information System Gastrointestinal Symptom Scales, 30-Second Chair Stand Test, Muscle Strength by Myometry Test, Newcastle Mitochondrial Disease Adult Scale, Newcastle Mitochondrial Disease Pediatric Scale, 36-Item Short Form Survey, Clinical Global Impression, Patient's Global Impression, Patient-Reported Initial MitoPC Symptoms, Patient-Reported MitoPC Symptom Changes, Columbia Suicide Severity Rating Scale, Neuropathy Impairment Score, Migraine Disability Assessment, Quick Inventory of Depressive Symptomatology—Self-Report, Montreal Cognitive Assessment, NY Heart Association Functional Classification, Diabetes Health Profile, Ocular Motility Assessments, Marginal Reflex Distance, Ocular Motility & Fixation in 8 Gaze Directions, Degree of Ocular Saccades, Visual Function Test, Logarithm of Minimum Angle of Resolution, Pelli-Robson Score, Humphrey Visual Field mean deviation, or a combination thereof. In preferred embodiments, the two or more measurement instruments are selected from Six Minute Walk Test, Motor Function Measure, Modified Fatigue Impact Scale, Friedreich's Ataxia Rating Scale and Patient-Reported Outcomes Measurement Information System Gastrointestinal Symptom Scales. In more preferred embodiments, the two or more measurement instruments are the Six Minute Walk Test, Motor Function Measure, Modified Fatigue Impact Scale, Friedreich's Ataxia Rating Scale and Patient-Reported Outcomes Measurement Information System Gastrointestinal Symptom Scales. Most of these measurement instruments, including how to conduct, measure, and evaluate them, are known in the art. Karaa et al., J. Inherit. Metab. Dis. 2017; 40(3):403-14.

In one aspect, the measurement instrument is a Motor Function Measure. In another aspect, the measurement instrument is a Six Minute Walk Test. In a further aspect, the measurement instrument is a Two Minute Walk Test. In one aspect, the measurement instrument is Modified Fatigue Impact Scale. In another aspect, the measurement instrument is a Friedreich's Ataxia Rating Scale. In a further aspect, the measurement instrument is Patient-Reported Outcomes Measurement Information System. In one aspect, the measurement instrument is a 30-Second Chair Stand Test. In another aspect, the measurement instrument is a Muscle Strength by Myometry Test. In another aspect, the measurement instrument is a Newcastle Mitochondrial Disease Adult Scale. In a further aspect, the measurement instrument is a Newcastle Mitochondrial Disease Pediatric Scale. In one aspect, the measurement instrument is a 36-Item Short Form Survey. In another aspect, the measurement instrument is a Clinical Global Impression. In one aspect, the measurement instrument is a Patient's Global Impression. In another aspect, the measurement instrument is a Patient-Reported Initial MitoPC Symptoms. In a further aspect, the measurement instrument is a Patient-Reported MitoPC Symptom Changes. In a further aspect, the measurement instrument is a Columbia Suicide Severity Rating Scale. In one aspect, the measurement instrument is a Neuropathy Impairment Score. In another aspect, the measurement instrument is a Migraine Disability Assessment Test. In a further aspect, the measurement instrument is a Quick Inventory of Depressive Symptomatology—Self-Report. In one aspect, the measurement instrument is a Montreal Cognitive Assessment. In another aspect, the measurement instrument is a NY Heart Association Functional Classification. In a further aspect, the measurement instrument is a Diabetes Health Profile. In one aspect, the measurement instrument is an Ocular Motility Assessment(s). In another aspect, the measurement instrument is a Marginal Reflex Distance. In a further aspect, the measurement instrument is an Ocular Motility & Fixation in 8 Gaze Directions. In one aspect, the measurement instrument is a Degree of Ocular Saccades. In another aspect, the measurement instrument is a Visual Function Test. In another aspect, the measurement instrument is a Logarithm of Minimum Angle of Resolution. In a further aspect, the measurement instrument is a Pelli-Robson Score. In one aspect, the measurement instrument is a Humphrey Visual Field mean deviation.

In some embodiments, the present techniques are referred to as a “MitoPC” (the Mitochondrial Disease Personalized Composite) approach. In some embodiments, two or more sub-instruments are selected from Motor Function Measure, Six Minute Walk Test, Two Minute Walk Test, Modified Fatigue Impact Scale, Friedreich's Ataxia Rating Scale, Patient-Reported Outcomes Measurement Information System Gastrointestinal Symptom Scales, Five Times Sit to Stand Test, Newcastle Mitochondrial Disease Adult Scale, Newcastle Mitochondrial Disease Pediatric Scale, 36-Item Short Form Survey, Clinical Global Impression, Patient's Global Impression, Patient-Reported Initial MitoPC Symptoms, Patient-Reported MitoPC Symptom Changes, Columbia Suicide Severity Rating Scale, Neuropathy Impairment Score, Migraine Disability Assessment Test, Quick Inventory of Depressive Symptomatology—Self-Report, Montreal Cognitive Assessment, NY Heart Association Functional Classification, Diabetes Health Profile, Ocular Motility Assessments, Marginal Reflex Distance, Ocular Motility & Fixation in 8 Gaze Directions, Degree of Ocular Saccades, Visual Function Test, Logarithm of Minimum Angle of Resolution, Pelli-Robson Score, Humphrey Visual Field mean deviation, or a combination thereof. One or more sub-instrument measurements can be obtained from each of the sub-instruments.

In some embodiments, the two or more sub-instrument measurements are validated outcome measures for mitochondrial dysfunction or mitochondrial disease.

In some embodiments, the methods disclosed herein are employed to diagnose, treat or manage subjects with mitochondrial dysfunction or mitochondrial disease.

“Treat,” “treatment,” and like terms refer to therapeutic treatment that reduces the severity and/or frequency of symptoms, eliminates symptoms and/or the underlying cause of the symptoms, reduces the frequency or likelihood of symptoms and/or their underlying cause, improves or remediates damage caused, directly or indirectly, by the mitochondrial dysfunction or mitochondrial disease, delays the onset of mitochondrial dysfunction or mitochondrial disease, or prevents the onset of mitochondrial dysfunction or mitochondrial disease. Subjects to be treated include those that have mitochondrial dysfunction or mitochondrial disease as well as those prone to develop mitochondrial dysfunction or mitochondrial disease. In certain embodiments, subjects with or prone to develop mitochondrial dysfunction or mitochondrial disease are clinically asymptomatic. Also within the scope of the disclosure are treatments that prevent the onset of symptoms in subjects in which mitochondrial dysfunction or mitochondrial disease is to be prevented. Those subjects in which mitochondrial dysfunction or mitochondrial disease is to be prevented can be ascertained using methods described herein. In further embodiments, treatments include those that prevent decompensation in the face of stressors or those that add resiliency in the face of stressors, as measured using acceptable clinical methods known to one of skill in the art.

In certain embodiments, the subject is diagnosed as having a genetic mitochondrial disease. In some aspects, the diagnosis of mitochondrial disease my involve cellular or tissue biopsy-based biochemical investigations or massively parallel DNA or RNA sequencing in blood and/or tissue.

In some aspects, the diagnosis is achieved by: (a) sequencing a plurality of nucleic acids obtained from a biological sample from the subject; (b) comparing the subject's nucleic acid sequence or a portion thereof to a plurality of reference sequences in a database comprising sequences containing pathologic nucleic acid mutations; and (c) determining whether the subject's nucleic acid sequence has at least one pathologic nucleic acid mutation in the database. In some embodiments, the database comprises sequences containing pathologic nucleic acid mutations refers to the Mitochondrial Disease Sequence Data Resource (MSeqDR), a community-wide Internet resource to curate mitochondrial disease genomic data. Falk et al., Mol. Genet. Metab. 2015; 114(3):388-396. As of 2019, the MSeqDR manages 267 mitochondrial diseases, 1,607 genes associated with mitochondrial biology or disease, and 4,486 pathogenic variants in those genes. Shen et al., Hum. Mutat. 2016; 37(6):540-548; see also MseqDR web site: mseqdr.org/mb.php?url=index.php. In some embodiments, the nucleic acids are DNA. In certain embodiments, the nucleic acid is RNA. In certain embodiments, the nature, abundance and location of chemical modifications to component nucleotides in the nucleic acid are employed for diagnostic purposes.

As of 2016, there were nearly 300 distinct genes in which mutations are known to cause mitochondrial disease (Gorman et al., Nat. Rev. Dis. Prim. 2016; 2:16080. Koopman et al., N. Engl. J. Med. 2012; 366:1132-41), with pathogenic etiologies arising in both the nuclear DNA genome and the mitochondrial DNA (mtDNA) genome. McCormick et al., Neurotherapeutics 2013; 10:251-61. In some embodiments, the at least one pathologic mutation is a substitution, deletion, or insertion.

In some embodiments, the at least one pathologic nucleic acid mutation is a mitochondrial DNA (mtDNA) mutation or a combination of mtDNA mutations. Exemplary mtDNA-encoded genes associated with mitochondrial disease when mutated include MT-ND1, MT-ND2, MT-ND3, MT-ND4, MT-ND4L, MT-ND5, MT-ND6, MT-CYB, MT-CO1, MT-CO2, MT-CO3, MT-RNR1, MT-RNR2, MT-TA, MT-TC, MT-TE, MT-TF, MT-TH, MT-TI, MT-TK, MT-TL1, MT-TL2, MT-TM, MT-TN, MT-TQ, MT-TS1, MT-TS2, MT-TV, MT-TW, MT-ATP6, and MT-ATP8, and combinations thereof. In certain embodiments, the mtDNA mutation is in a gene for a mitochondrial transfer RNA (mt-tRNA) or in a gene for a mitochondrial ribosomal RNA (mt-rRNA).

In some embodiments, the at least one pathologic nucleic acid mutation is a nuclear DNA (nDNA) mutation or a combination of nDNA mutations. Exemplary nDNA-encoded genes associated with mitochondrial disease when mutated include, but are not limited to, AARS2, ABCB7, ABCC8, ACAD8, ACAD9, ACADM, ACADS, ACADSB, ACADVL, ACAT1, ACO2, ADCK3, ADRB2, ADRB3, AFG3L2, AGK, AGRP, AIFM1, AK2, AKAP10, AKT2, ALAS2, ALDH2, ALDH4A1, ALDH6A1, AMACR, AMT, APOPT1, APTX, ARMS2, ATP5A1, ATP5E, ATP5F1A, APT5F1D, ATP5F1E, ATPAF2, AUH, BAX, BCAT2, BCKDHA, BCKDHB, BCL2, BCS1L, BOLA3, C8orf38, C10orf2, C12orf62, C12orf65, C19orf12, C20orf7, C1QBP, CAPN10, CARS2, CARTPT, CDH23, CDKAL1, CHCHD10, CHKB, CISD2, CLRN1, COA5, COA7, COA10, COQ2, COQ4, COQ6, COQ7, COQ8A, COQ9, COX10, COX14, COX15, COX20, COX412, COX6B1, COX8A, CPS1, CPT1A, CPT2, CRAT, CYB5R3, CYC1, CYC5, CYCS, CYP11A1, CYP11B1, CYP11B2, CYP24A1, CYP27A1, CYP27B1, D2HGDH, DARS2, DBT, DECR1, DFNB31, DGUOK, DHODH, DIABLO, DLD, DLAT, DMGDH, DNA2, DNAJC19, DNM1L, EARS2, ECHS1, ELAC2, ENPP1, ETFA, ETFB, ETFDH, ETHE1, FARS2, FASTKD2, FBXL4, FOXRED1, FH, FXN, GATM, GCDH, GCGR, GCK, GCSH, GDAP1, GFER, GFM1, GHRL, GJB2, GJB3, GJB6, GK, GLDC, GLRX5, GLUD1, GPD2, GPR98, GTPBP3, HADH, HADHA, HADHB, HARS2, HCCS, HIBCH, HK1, HLCS, HMGA1, HMGCS2, HMGCL, HNF1A, HNF1B, HNF4A, HOGA1, HSD17B10, HSPD1, HTRA1, HTRA2, IBA57, IDH2, IDH3B, IGF2BP2, IL6, INSR, IRS1, IRS2, ISCA1, ISCA2, ISCU, IVD, KANK1, KARS, KCNJ11, KIF1B, L2HGDH, LARS2, LEPR, LIAS, LIPC, LRPPRC, LRRK2, LYRM4, LYRM7, MAOA, MAPK8IP1, MARS2, MC4R, MCCC1, MCCC2, MCEE, ME2, MFF, MFN2, MGME1, MIPEP, MLYCD, MMAA, MMAB, MMADHC, MPC1, MPV17, MRAP2, MRPL3, MRPL44, MRPS2, MRPS7, MRPS16, MRPS22, MRPS34, MSTO1, MTFMT, MTO1, MTPAP, MUT, MYO7A, NAGS, NARS2, NDUFA1, NDUFA2, NDUFA6, NDUFA9, NDUFA10, NDUFA11, NDUFA12, NDUFA13, NDUFAF1, NDUFAF2, NDUFAF3, NDUFAF4, NDUFAF5, NDUFAF6, NDUFB3, NDUFB8, NDUFB9, NDUFS1, NDUFS2, NDUFS3, NDUFS4, NDUFS5, NDUFS6, NDUFS7, NDUFS8, NDUFV1, NDUFV2, NEUROD1, NF2, NFU1, NGLY1, NROB2, NUBPL, NUP62, OAT, OGDH, OPA1, OPA3, OTC, OXCT1, PANK2, PARK2, PARK7, PAX4, PC, PCCA, PCCB, PCDH15, PCK2, PDHA1, PDHB, PDHX, PDP1, PDSS1, PDSS2, PDX1, PET100, PINK1, PNKD, PNPT1, POLG, POLG2, POMC, PPARG, PPARGC1B, PPOX, PPP1R3A, PUS1, PYCR1, PYY, RARS2, REEP1, RMRP, RMND1, RNASEH1, RRM2B, SACS, SARDH, SARS2, SCO1, SCO2, SCO3, SDHA, SDHAF1, SDHAF2, SDHB, SDHC, SDHD, SERAC1, SFXN4, SIM1, SLC2A2, SLC2A4, SLC22A5, SLC25A1, SLC25A3, SLC25A4, SLC25A12, SLC25A13, SLC25A15, SLC25A19, SLC25A20, SLC25A22, SLC25A26, SLC25A38, SLC30A8, SOD2, SPG7, SPG20, STAR, SUCLA2, SUCLG1, SURF1, TACO1, TARS2, TAZ, TDF7L2, TFAM, TIMM8A, TIMMDC1, TK2, TMEM70, TMEM126, TMEM126B, TOP3A, TRIT1, TRMT10C, TRMT5, TRMU, TRNT1, TSFM, TTC19, TUFM, TWNK, UCP1, UCP2, UCP3, UNG, UQCC2, UQCC3, UQCRB, UQCRC2, UQCRQ, USMG5, USH1C, USH1G, USG2A, VARS2, WARS2, WFS1, WWOX, XPNPEP3, YARS2, and combinations thereof.

In some aspects, the pathologic nucleic acid mutation is a combination of at least one mtDNA mutation and at least one nDNA mutation, and/or are dependent on mtDNA haplogroup background and/or environmental exposure.

In certain aspects of the disclosure, the mitochondrial dysfunction is associated with neurodegenerative disorders such as Parkinson's disease, amyotrophic lateral sclerosis, multiple sclerosis, or Huntington's disease. In some aspects of the disclosure, the mitochondrial dysfunction is associated with cognition, psychiatric, and/or mood impairment. In further aspects of the disclosure, the mitochondrial dysfunction is associated with Alzheimer's disease. In still further aspects of the disclosure, the mitochondrial dysfunction is associated with cardiac dysfunction.

In some embodiments, the subject is any type of subject of a suitable age. As used herein, “subject” includes mammals, in particular, domesticated animals such as dogs, cats, horses, pigs, sheep, cattle and the like. In preferred embodiments, “subject” includes humans. The terms “human,” “patient,” and “subject” are used interchangeably herein.

In certain embodiments, the subject is human of 18 years or older. In some embodiments, the subject is a child that is less than 18 years old. In some embodiments, the subject is a fetus, newborn, or infant. In some aspects, the subject is an adolescent. In certain embodiments, the subject is between about 3 weeks to about 52 weeks of age. In preferred embodiments, the subject is 18 years or older, 17 years or younger, 12 years or younger, 65 years or younger, 75 years or younger, or between about 3 weeks to about 52 weeks of age.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

It is to be appreciated that certain features of the disclosed methods and systems which are, for clarity, described herein in the context of separate embodiments, may also be provided in combination in a single embodiment. Conversely, various features of the disclosed methods that are, for brevity, described in the context of a single embodiment, may also be provided separately or in any sub-combination.

The term “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. For example, the range of “1 mg to about 10,000 mg” is inclusive of the endpoints, 1 mg and 10,000 mg, and all the intermediate values. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

### EXAMPLES

The following examples are provided to illustrate some of the embodiments disclosed herein. The examples are intended to illustrate, not to limit, the disclosed embodiments.

### Example 1. Computation of a Composite Measurement

A non-limiting example of computation of a composite measurement in subjects with genetically-confirmed mitochondrial disease is described below. As mentioned above, in some embodiments, the present approach is referred to as “MitoPC.”

The five exemplary MitoPC sub-instruments, and the associated clinical symptoms assessed, are summarized in Table 1 below.

TABLE 1
MitoPC
MitoPC: symptoms assessed and sub-instruments

| Symptom | Sub-Instrument | Code |
|---|---|---|
| Exercise intolerance | 6-Minute Walk Test | 6MWT |
| Muscle weakness | Motor Function Measure (32 items) | MFM-32 |
| Fatigue | Modified Fatigue Impact Scale | MFIS |
| Balance problems | Friedreich's Ataxia Rating Scale | FARS |
| Gastrointestinal | Patient-Reported Outcomes Measurement Information | PROMIS-GI |
| symptoms | System-Gastrointestinal Symptom Scales | |

A plurality of MitoPC sub-instruments may be administered to each subject of a plurality of subjects, but only “qualified sub-instruments” (where the initial sub-instrument result meets or exceeds a pre-defined threshold value) are included in the MitoPC analysis. For example, in some embodiments, all sub-instruments may be administered to each subjects, but only a certain number of the sub-instruments are selected for further analysis.

The sub-instruments may be administered to the subject at a single point in time or over a time period (e.g., a treatment time period), such that a plurality of measurements are obtained for each sub-instrument. Each subject is classified as a “Sub-instrument Responder” or “Sub-instrument Non-responder” for each “qualified sub-instrument” during each treatment period, by comparing the measured change to an established Minimum Clinically Important Difference (“MCID” or “M”) for that sub-instrument (Table 2). In some embodiments, the MCID for each MitoPC sub-instrument is used to define clinically-significant changes in the subject's condition after drug intervention (e.g., improvement or deterioration in the subject's condition).

TABLE 2
MitoPC Sub-Instrument Responders & Non-Responders

Change relative to baseline

| | | | |
|---|---|---|---|
| Change | ≤−M | >−M and <+M | ≥+M |
| Assessment | Worse | Neutral | Better |

| | | |
|---|---|---|
| Definition | Sub-instrument Non-Responder | Sub-instrument Responder |

(M = MCID)

As shown in Table 2, a “Sub-instrument Responder” is defined as a subject that achieves an improvement over a baseline that equals or exceeds the MCID for that sub-instrument (i.e., an assessment of “Better”). The baseline may be a baseline defined for a single point or a baseline defined over a treatment period.

If a subject does not achieve an MCID improvement for that sub-instrument (e.g., the assessment results in “Neutral” or “Worse” assignments), then that subject is considered a “Sub-instrument Non-responder”. The baseline may be a baseline defined for a single point or a baseline defined over a treatment period. Each of the various sub-instruments that can be employed in accordance with embodiments of the present disclosure can have different baseline(s) defined for a certain sub-instrument. A different between a sub-instrument measurement and the baseline is compared to a MCID, to determine if there was an improvement, deterioration, or no change in the measurements, as compared to the baseline.

Furthermore, the subject will then be categorized as a “MitoPC Responder” or a “MitoPC Non-Responder.” A “MitoPC Responder” is a subject who is a “Sub-instrument Responder” for at least one of the “qualified sub-instruments” in that subject's personalized MitoPC, in the absence of a clinically meaningful deterioration in any of the other “qualified sub-instruments” (Table 3).

A “MitoPC Non-Responder” is a subject who is not a “Sub-instrument Responder” for at least one of the “qualified sub-instruments” in that subject's personalized MitoPC, or experiences a clinically meaningful deterioration in any of the other “qualified sub-instruments” (Table 3). In some embodiments, five or fewer sub-instruments may be selected as “qualified sub-instruments,” though it should be appreciated that other number of qualified sub-instruments can be used.

TABLE 3
MitoPC Responder & Non-Responder Definitions
MitoPC Sub-Instruments

| # | Symptom | Sub-Instrument | Assessment | Definition |
|---|---|---|---|---|
| 1 | Exercise | Six-Minute Walk Test | Better | 6MWT Responder |
| | intolerance | | Neutral or Worse | 6MWT Non-Responder |
| 2 | Muscle | Motor Function Measure | Better | MFM-32 Responder |
| | weakness | | Neutral or Worse | MFM-32 Non-Responder |
| 3 | Fatigue | Modified Fatigue Impact Scale | Better | MFIS Responder |
| | | | Neutral or Worse | MFIS Non-Responder |
| 4 | Balance | Friedreich's Ataxia Rating Scale | Better | FARS Responder |
| | problems | | Neutral or Worse | FARS Non-Responder |
| 5 | GI problems | Patient-Reported Outcomes | Better | PROMIS-GI Responder |
| | | Measurement Information System - | Neutral or Worse | PROMIS-GI Non-Responder |
| | | Gastrointestinal Symptoms | | |

| | |
|---|---|
| MitoPC Responder | Responder on any of the “qualified sub-instruments”, with no assessment of |
| | Worse for any of the “qualified sub-instruments” |
| MitoPC Non-Responder | Non-Responder on all of the “qualified sub-instruments”, or assessment of |
| | Worse for any of the “qualified sub-instruments” |

MitoPC Sub-Instrument Threshold Values are used in the described approach. For each sub-instrument, the threshold value is defined as a score that corresponds to a “mild” disease or symptom severity rating in literature, or the clinical equivalent thereof. These values were determined by a literature review and evaluation by a committee of mitochondrial disease clinicians and experts. Brennan et al., Am. J. Gastroenterol. 2014; 109:1804-1814. Flachenecker et al., Multiple Sclerosis 2002; 8:523-526. Subramony et al., Neurology 2005; 64:1261-1262. Berard et al., Neuromuscular Disorders 2005; 15:463-470. Enright and Scherrill, Am. J. Respir. Crit. Care Med. 1998; 158:1384-1387.

These threshold values are employed for patient qualification and statistical analysis of the MitoPC endpoint, and are not necessarily considered in the clinical administration of the subsequent sub-instrument tests.

Scores or values below threshold indicate values that are typical of asymptomatic individuals and the general population. The derived threshold values for each sub-instrument are stated in Table 4.

TABLE 4
MitoPC Sub-Instrument Threshold Values

| | Sub-Instrument | Threshold Value | | |
|---|---|---|---|---|
| 1 | 6MWT | Less then | (determined for a | Meters |
| | | | subject)** | |
| 2 | MFM-32 | Greater than or equal to | 89 | Points |
| 3 | MFIS | Greater than or equal to | 39 | Points |
| 4 | FARS | Greater than or equal to | 29 | Points |
| 5 | PROMIS-GI | Greater than or equal to | 55 | T-score Points |

**6MWT is determined for each subject, based on the subject's gender, age, height and weight.

### Statistical Methods

Data will be collected and processed according to the Standard Operating Procedures of the Sponsor's Clinical Research Organization, which are based on the principles of Good Clinical Practices.

MitoPC Statistical Methods: the primary assessment is the comparison of treatment with an Active versus Placebo in the proportion of “MitoPC Responders” as previously defined.
Specifically, if: P_{Active}=Probability (MitoPC Responder, Active)
   P_{Placebo}=Probability (MitoPC Responder, Placebo)
then the primary hypothesis test of interest is: H₀: P_{Active}=P_{Placebo}
   HA: P_{Active}≠P_{Placebo}.

The “MitoPC Responder” definition allows the results of the study to be reduced to a dichotomous outcome: Responder vs. Non-Responder. These dichotomous outcomes can be summarized in the following contingency table (Table 5).

TABLE 5
Contingency Table

| | | Active Treatment | | |
|---|---|---|---|---|
| | | Responder | Non-Responder | |
| Placebo Treatment | Responder | a | b | a + b |
| | Non-Responder | c | d | c + d |

| | | |
|---|---|---|
| a + c | b + d | a + b + c + d = n |

The proportion of subjects given Active treatment that are “MitoPC Responders” is (a+c)/n. Similarly, the proportion of subjects given Placebo treatment that are “MitoPC Responders” is (a+b)/n. Since “a” is common to both proportions, the “b” and “c” discordant pairs are critical to the test of the primary hypothesis.

The McNemar's test is used to test the null hypothesis of whether the probability of a subject being a “MitoPC Responder” after treatment with an Active, is the same as the probability of being a “MitoPC Responder” after treatment with placebo. Specifically, the McNemar's exact binomial test will be used for these calculations, since the expected number of discordant pairs may be relatively small (<25).

Sample Size: The sample size calculation for McNemar's exact test is based on the expected proportions of subjects allocated into the discordant pair categories described above (b/n and c/n). For example, assuming that 40% of the subjects will be classified as an “Active Responder/Placebo Non-Responder”, and 5% of the subjects will be classified as an “Active Non-Responder/Placebo Responder”, then at least 30 subjects being evaluated need to complete the study in order to ensure at least 85% power for testing the null hypothesis, given a Type I error rate of 0.05.

Assuming an attrition rate of no more than 32%, 44 subjects will need to be enrolled/randomized for a minimum of 30 evaluable subjects (i.e., subjects that complete the entire study).

Analysis Populations: All analyses will be performed for at least one of the following analysis populations:
Intent-to-Treat (ITT) Population: All subjects who receive at least one dose of the study drug and have at least one post-baseline efficacy data point will be included in the ITT population. Subject data will be included with the treatment allocation to which they were randomized.
Per-Protocol (PP) Population: Subjects included in the ITT population who have no protocol deviations or missing data that are considered meaningful to the interpretation of the endpoint will be included in the PP population.
Safety Population: All subjects who receive at least one dose of a drug (including placebo) will be included in the safety evaluations.
   The frequencies of AEs by type, body system, severity, and relationship to study drug will be summarized. SAEs, if any, will be described in detail. AE incidence will be summarized with the corresponding exact binomial 95% two-sided confidence intervals.

1. A method of computing a composite measurement for the assessment, diagnosis, monitoring, or treatment of a subject with mitochondrial dysfunction or mitochondrial disease, said method comprising:
(i) employing a panel of two or more sub-instruments to measure one or more clinical symptoms of mitochondrial dysfunction or mitochondrial disease in a subject,
(ii) combining the measurements obtained from said two or more sub-instruments into a single composite measurement, and
(iii) assessing the overall severity of, or change in, the mitochondrial dysfunction or mitochondrial disease in the subject by comparing the composite measurement to a reference value or another composite measurement in the same subject.

2. The method of claim 1, wherein:
(i) one or more composite measurements are employed to measure the clinical effect on the subject of a diagnostic, therapeutic or other type of medical intervention;
(ii) for each of the sub-instruments in said panel, the subject is classified as:
(a) a sub-instrument responder or sub-instrument non-responder,
(b) a member of a clinical category, or
(c) a member of a metric range, based on the change in said one or more clinical symptoms as measured using said sub-instrument; and
(iii) the measurements obtained from the sub-instruments in the panel are combined into a single composite measurement, by either:
(a) separately assessing the change in each measurement obtained from the panel sub-instruments prior to combining each measurement into a single composite measurement, or
(b) combining measurements obtained from the panel sub-instruments at a first time point and generating a single composite measurement for said first time point and then comparing the single composite measurement for said first time point to a single composite measurement generated from the same panel sub-instruments for a second time point.

3. The method of claim 1, wherein the panel sub-instruments comprise one or more of Motor Function Measure, Six Minute Walk Test, Two Minute Walk Test, Modified Fatigue Impact Scale, Friedreich's Ataxia Rating Scale, Patient-Reported Outcomes Measurement Information System Gastrointestinal Symptom Scales, 30-Second Chair Stand Test, Muscle Strength by Myometry Test, Newcastle Mitochondrial Disease Adult Scale, Newcastle Mitochondrial Disease Pediatric Scale, 36-Item Short Form Survey, Clinical Global Impression, Patient's Global Impression, Patient-Reported Initial MitoPC Symptoms, Patient-Reported MitoPC Symptom Changes, Columbia Suicide Severity Rating Scale, Neuropathy Impairment Score, Migraine Disability Assessment Test, Quick Inventory of Depressive Symptomatology—Self-Report, Montreal Cognitive Assessment, NY Heart Association Functional Classification, Diabetes Health Profile, Ocular Motility Assessments, Marginal Reflex Distance, Ocular Motility & Fixation in 8 Gaze Directions, Degree of Ocular Saccades, Visual Function Test, Logarithm of Minimum Angle of Resolution, Pelli-Robson Score, Humphrey Visual Field mean deviation, or a combination thereof.

4. The method of claim 1, wherein the panel sub-instruments comprise Motor Function Measure, Six Minute Walk Test, Modified Fatigue Impact Scale, Friedreich's Ataxia Rating Scale, and the Patient-Reported Outcomes Measurement Information System Gastrointestinal Symptom Scales.

5. The method of claim 1, wherein the panel sub-instruments comprises five sub-instruments.

6. The method of claim 5, wherein the five sub-instruments comprise Motor Function Measure, Six Minute Walk Test, Modified Fatigue Impact Scale, Friedreich's Ataxia Rating Scale, and the Patient-Reported Outcomes Measurement Information System Gastrointestinal Symptom Scales.
